(19) United States
(12) Patent Application Publication
(10) Pub. No.: US 2006/0039985 A1
(43) Pub. Date: Feb. 23, 2006

(54) Methotrexate compositions

(76) Inventors: David B. Bennett, San Jose, CA (US); David Lechuga-Ballesteros, Santa Clara, CA (US)

Correspondence Address: NEKTAR THERAPEUTICS, 150 INDUSTRIAL ROAD, SAN CARLOS, CA, 94070, US

(21) Appl. No.: 11/115,776

(22) Filed: Apr. 27, 2005

Related U.S. Application Data
(60) Provisional application No. 60/565,992, filed on Apr. 27, 2004.

(51) Int. Cl. A61K 31/525; A61K 9/14

(52) U.S. Cl. 424/489; 514/251

(57) ABSTRACT

A powder containing methotrexate particles in liquid crystal form suitable for inhalation.

### RELATION TO PREVIOUS APPLICATIONS

This application claims the benefit of provisional application 60/565,992 filed 27 Apr. 2004.

### FIELD OF THE INVENTION

The present invention relates to the liquid crystal form of methotrexate and to methotrexate-containing particles. A plurality of methotrexate-containing particles can result in powdered compositions. In addition, the invention relates to methods for making and administering compositions comprising methotrexate.

### BACKGROUND OF THE INVENTION

Methotrexate and its synthetic preparation is described in U.S. Pat. No. 2,512,572. Methotrexate is a well known anti cancer agent that acts by inhibition of the ubiquitous enzyme dihydrofolate reductase. Methotrexate is also known to have potent anti-inflammatory activity. Methotrexate is used as a therapeutic for a variety of ailments such as leukemia, choriocarcinoma in women, psoriasis, and various cancers, including lung cancer. Methotrexate is chemically classified as a pteridine, pharmacologically classified as a folic acid antagonist, and therapeutically classified as an antineoplastic and antirheumatic.

Methotrexate is currently available as tablets (sodium salt), solution (for injection), and lyophilized powder for injection. The currently available forms and formulations of methotrexate, however, suffer from a limited potential. For example, the currently available crystalline forms of methotrexate may be prone to physical instability. Alternative forms of methotrexate have the potential to alleviate or reduce the problems associated with physical instability.

Methotrexate, derivatives, formulations and applications have been described in a number of publications. U.S. Pat. No. 6,559,149 describes methotrexate derivatives useful as lymphocyte production suppressants, for treating rheumatism. U.S. Pat. No. 5,958,928 describes various formulations containing methotrexate derivatives for the treatment of lupus. U.S. Pat. No. 5,728,692 describes methotrexate derivatives with reduced toxicity for the treatment of arthritis. U.S. Pat. No. 5,292,731 describes the use of methotrexate for the treatment of hyper-proliferative skin disease; particularly employing a zinc salt of methotrexate applied topically which is claimed to have improved penetration and lower toxicity.

Various types of solid formulation of methotrexate suitable for pulmonary delivery in dry powder form are described by Hak-Kim Chan and Igor Gondor in Methotrexate: Existence Of Different Types Of Solid, Int. J. Pharmaceutics, 68 (1991) 197-190.

Liquid crystal forms of methotrexate have been discussed in a publication by the applicants (David Lechuga-Ballesteros et al., Liquid Crystallinity Improves the Physical Stability of Spray Dried Powders for Inhalation, Resp. Drug Delivery IX, 2004, pp. 569-572).

The present invention is directed to methotrexate forms and formulations that solve various problems associated with physical instability and a relative dearth of dosing options. Current formulations of methotrexate disclosed herein include spray-dried particle formulations that may be administered by inhalation.

### SUMMARY OF THE INVENTION

Accordingly, it is a primary object of the invention to provide a spray-dried particle comprising methotrexate.

It is a further object of the invention to provide methotrexate in liquid crystal form.

It is an additional object of the invention to provide compositions comprised of a plurality of spray-dried particles comprised of methotrex ate.

It is yet a further object of the invention to provide such compositions further comprising a pharmaceutically acceptable excipient.

It is still further object of the invention to provide a method for administering an methotrexate to the lungs of a patient comprising the steps of: (i) providing a formulation comprised of methotrexate and suited for delivery via inhalation; (ii) dispersing the formulation to form an aerosol; and (iii) delivering the aerosol to the lungs of the patient by inhalation of the aerosol by the patient, thereby ensuring delivery of methotrexate to the lungs of the patient.

It is also an object of the invention to provide a method for preparing a methotrexate-containing formulation comprising the steps of: (i) combining methotrexate, an optional excipient, and solvent to form a mixture or solution; and (ii) spray drying the mixture or solution to obtain dry, particles comprised of methotrexate and the excipient, when present.

It is still yet a further object of the invention to provide methotrexate particles (optionally in the form of a powder formulation) that are suited for pulmonary administration.

Additional objects, advantages and novel features of the invention will be set forth in the description that follows, and in part, will become apparent to those skilled in the art upon the following, or may be learned by practice of the invention.

The present invention is based, in part, upon the unexpected discovery of chemically and physically stable spray-dried powder formulations of methotrexate. Surprisingly, the spray-dried powders of the invention (comprised of a plurality of spray-dried particles) comprise methotrexate in a liquid crystalline form.

The methotrexate formulations, demonstrating insignificant degradation upon preparation and storage, may be prepared in the absence of stabilizing additives or excipients, or may further include a pharmaceutically acceptable excipient. Preferred excipients include sucrose, mannitol, citrate (e.g., sodium citrate or potassium citrate), leucine, trileucine, and combinations thereof. A more complete discussion of pharmaceutically acceptable excipients suited for inclusion in the present formulations is provided infra.

Additionally, the methotrexate powder compositions of the invention comprise particles effective to penetrate into the alveoli of the lungs, that is, having in a particular embodiment, a mass median diameter (MMD) of less than about 10 μm, preferably less than about 7.5 μm, and most preferably less than 5 μm in diameter. In a particularly preferred embodiment, the powder is composed of particles having an MMD from about 1.0 to 5.0 μm.

Further embodiments of the methotrexate powder compositions in accordance with the invention include spray-dried methotrexate particles having a mass median aerodynamic diameter (MMAD) of less than about 10 microns, preferably less than about 5.0 microns, and more preferably less than about 3.5 microns. In an especially preferred embodiment, the MMAD ranges from 1.5 to 3.5 microns.

Also encompassed by the invention is an aerosolized methotrexate powder formulation, and a methotrexate powder in a unit dosage form.

In another embodiment, the invention is directed to a method for administering a methotrexate powder composition as described herein to the lungs of a patient in need thereof. In the method, a composition as described above is administered by inhalation in aerosolized form.

The invention also encompasses, in yet another embodiment, a method for preparing a dispersible, dry methotrexate powder composition having the features described above.

In one embodiment, the respirable methotrexate powder composition is prepared by combining the methotrexate [and any other optional active agent(s)] in a suitable solvent to form a mixture or solution and spray drying the mixture or solution to obtain discrete, substantially amorphous particles, preferably in the form of a dry powder. The methotrexate remains essentially intact upon spray drying.

An optional pharmaceutical excipient may be further added to the solvent to form a homogeneous solution or heterogeneous mixture, such that spray drying of the solution or mixture produces particles comprising, in combination with methotrexate, excipient, buffer, and any other components that are present in the solution or mixture. Alternatively, the pharmaceutical excipient may be separately dissolved and spray dried to yield separate yet co-administrable powder particles.

### BRIEF DESCRIPTION OF THE DRAWINGS

FIG. 1 shows spray dried methotrexate as presenting an XRPD pattern (left) characteristic of an amorphous solid, in contrast to the crystalline raw material, but displays direfringence under polarized light (right) suggestive of a crystalline molecular order.

FIG. 2A show scanning electron micrographs (at various resolutions) of raw material methotrexate.

FIG. 2B show scanning electronic micrographs (at various resolutions) of spray dried methotrexate compositions.

FIGS. 3A, 3B and 3C are MDSC thermograms as discussed in the examples and figure page.

### DETAILED DESCRIPTION OF THE INVENTION

Before describing the present invention in detail, it is to be understood that this invention is not limited to the excipients, spray-drying methods, and the like as such may vary. It is also to be understood that the terminology used herein is for describing particular embodiments only, and is not intended to be limiting.

It must be noted that, as used herein, the singular forms “a,” “an,” and “the” include plural referents unless the context clearly dictates otherwise. Thus, for example, reference to an “excipient” includes a single excipient as well as two or more of the same or different excipients, and the like.

In describing and claiming the present invention, the following terminology will be used in accordance with the definitions described below.

The term “amino acid” refers to any molecule containing both an amino group and a carboxylic acid group and can serve as an excipient. Although the amino group most commonly occurs at the beta position (i.e., the second atom from the carboxyl group, not counting the carbon of the carboxyl group) to the carboxyl function, the amino group can be positioned at any location within the molecule. The amino acid can also contain additional functional groups, such as amino, thio, carboxyl, carboxamide, imidazole, and so forth. As used herein, the term “amino acid” specifically includes amino acids as well as derivatives thereof such as, without limitation, norvaline, 2-aminoheptanoic acid, and norleucine. The amino acid may be synthetic or naturally occurring, and may be used in either its racemic or optically active (D-, or L-) forms, including various ratios of stereoisomers. The amino acid can be any combination of such compounds. Most preferred are the naturally occurring amino acids. The naturally occurring amino acids are phenylalanine, leucine, isoleucine, methionine, valine, serine, proline, threonine, alanine, tyrosine, histidine, glutamine, asparagines, lysine, aspartic acid, glutamic acid, cysteine, tryptophan, arginine, and glycine.

By “oligopeptide” is meant any polymer in which the monomers are amino acids totaling generally less than about 100 amino acids, preferably less than 25 amino acids. The term oligopeptide also encompasses polymers composed of two amino acids joined by a single amide bond as well as polymers composed of three amino acids.

“Dry” when referring to a powder (e.g., as in “dry powder”) is defined as containing less than about 10% moisture. Preferred compositions contain less than 7% moisture, more preferably less than 5% moisture, even more preferably less than 3% moisture, and most preferably less than 2% moisture. The moisture of any given composition can be determined by, for example, the Karl Fischer titrimetric technique using a Mitsubishi moisture meter model # CA-06. Thermogravimetric analysis (“TGA”) can also be used.

As used herein, an “excipient” is a non-methotrexate component of a particle, powder or composition intended to be in the particle, powder or composition. Thus, “excipients” such as buffers, sugars, amino acids, and so forth are intended components of a formulation and stand in contrast to unintended components of a formulation such as impurities (e.g., dust) and the like.

A “therapeutically effective amount” is the amount of methotrexate required to provide a desired therapeutic effect. The exact amount required will vary from subject to subject and will otherwise be influenced by a number of factors, as will be explained in further detail below. An appropriate “therapeutically effective amount,” however, in any individual case can be determined by one of ordinary skill in the art.

The term “substantially” refers to a system in which greater than 50%, more preferably greater than 85%, still more preferably greater than 92%, and most preferably greater than 96%, of the stated condition is satisfied.

The term “patient” refers to a living organism suffering from or prone to a condition that can be prevented or treated by administration of methotrexate, and includes both humans and animals.

“Optional” and “optionally” means that the subsequently described circumstance may or may not occur, so that the description includes instances where the circumstance occurs and instances where it does not. Thus, for example, a formulation comprising an “optional excipient” includes formulations comprising one or more excipients as well as formulations lacking any excipient.

Compositions of the present invention are considered to be “respirable” if they are suitable for inhalation therapy (i.e., capable of being inspired by the mouth or nose and drawn through the airways and into the lungs) and/or pulmonary delivery (i.e., local delivery to the tissues of the deep lung and optionally absorption through the epithelial cells therein into blood circulation). Compositions of the present invention can provide for rapid action, providing, for example, therapeutically effective levels locally (e.g., at local pulmonary tissues) and/or systemically (e.g., within the systemic circulation) in less than 60 minutes. Advantageously with respect to the treatment of lung cancer and other diseases of the lung, the present compositions are effective without the need to obtain systemic circulation.

“Orally respirable” compositions are those respirable compositions that are particularly adapted for oral inhalation. Likewise, “nasally respirable” compositions are those respirable compositions that are particularly adapted for nasal inhalation, i.e., intranasal delivery into the upper respiratory tract.

“Emitted Dose” or “ED” provides an indication of the delivery of a drug formulation from a suitable inhaler device after a firing or dispersion event. More specifically, for dry powder formulations, the ED is a measure of the percentage of powder that is drawn out of a unit dose package and which exits the mouthpiece of an inhaler device. The ED is defined as the ratio of the dose delivered by an inhaler device to the nominal dose (i.e., the mass of powder per unit dose placed into a suitable inhaler device prior to firing). The ED is an experimentally determined parameter, and is typically determined using an in vitro device arranged to mimic patient dosing. To determine an ED value, a nominal dose of dry powder, typically in unit dose form, is placed into a suitable dry powder inhaler (such as described in U.S. Pat. No. 5,785,049) and then actuated, dispersing the powder. The resulting aerosol cloud is then drawn by vacuum from the device, where it is captured on a tared filter attached to the device mouthpiece. The amount of powder that reaches the filter constitutes the emitted dose. For example, for a 5 mg, dry powder-containing dosage form placed into an inhalation device, if dispersion of the powder results in the recovery of 4 mg of powder on a tared filter as described above, then the emitted dose for the dry powder composition is: 4 mg (delivered dose)/5 mg (nominal dose)×100=80%. For nonhomogenous powders, ED values provide an indication of the delivery of drug from an inhaler device after firing rather than of dry powder, and are based on amount of drug rather than on total powder weight. Similarly for MDI and nebulizer dosage forms, the ED corresponds to the percentage of drug which is drawn from a unit dosage form and which exits the mouthpiece of an inhaler device.

As used herein, a “dispersible” powder is one having an ED value of at least about 5% preferably at least about 10%, more preferably at least about 40%, even more preferably at least about 55%, with ED values at least about 70% being most preferred.

“Mass median diameter” or “MMD” is a measure of mean particle size, since the powders of the invention are generally polydisperse (i.e., consist of a range of particle sizes). MMD values as reported herein are determined by centrifugal sedimentation, although any number of commonly employed techniques can be used for measuring mean particle size (e.g., electron microscopy, light scattering, laser diffraction. Typically, the MMD will be from about 0.5 to about 10 microns, more preferably from 1 to about 5 microns.

“Mass median aerodynamic diameter” or “MMAD” is a measure of the aerodynamic size of a dispersed particle. The aerodynamic diameter is used to describe an aerosolized powder in terms of its settling behavior, and is the diameter of a unit density sphere having the same settling velocity, in air, as the particle. The aerodynamic diameter encompasses particle shape, density and physical size of a particle. As used herein, MMAD refers to the midpoint or median of the aerodynamic particle size distribution of an aerosolized powder determined by cascade impaction, unless otherwise indicated.

“Fine Particle Fraction'as in “FPF_{<3.3 μm}” or “FPF_{<4.7 μm}” is defined as the amount of particles in a powder that are under 3.3 microns or 4.7 microns, respectively, as determined by cascade impaction. With respect to FPF_{<3.3 μm}, this parameter corresponds to the total mass under stage 3 of an Andersen impactor when operated at a flow rate of 1 cfm (28.3 L/min). The actual mass of particles satisfying the stipulated size range in a given amount of powder can be calculated and is abbreviated “FPM”

“Bulk density” refers to the density of a powder prior to compaction (i.e., the density of an uncompressed powder), and is typically measured by a well-known USP method. Typically, the compositions described herein will have a bulk density of from 0.01 to 10 grams per cubic centimeter.

The present invention provides, among other things, a novel liquid crystal form of methotrexate. It has unexpectedly been found that spray drying mixtures (preferably organic solutions) containing comprising methotrexate results in this novel form of methotrexate. Spray drying of mixtures comprising methotrexate produces powders where the particulate methotrexate so produced exhibits a lack of three-dimensional order as determined by powder X-ray diffraction (PXRD) and also exhibits two-dimensional order when analyzed by small angle X-ray scattering (SAXS). Further, the spray-dried methotrexate exhibits a phase change from solid to liquid over a narrow temperature range with a step-wise change in heat capacity, i.e., a glass transition-like melt. This form of methotrexate is liquid crystal methotrexate. The spray drying process conditions may be varied within certain limits to achieve very narrow particle size distributions that make the resulting powders especially suitable for efficient delivery by oral inhalation. These powders have high delivery efficiency when aerosolized with a dry powder inhaler and have demonstrated physical, chemical, and aerosol stability over prolonged periods of high temperature and humidity.

Amorphous materials, unlike liquid crystals, have no peaks by SAXS and are not birefringent to polarized light. Liquid crystals show a distinct melt over a narrow temperature range unlike amorphous glasses which show no such melt.

Thus, in one aspect, the invention provides liquid crystal methotrexate, in particular thermotropic liquid crystal methotrexate.

Turning to another aspect of the invention, highly dispersible powder compositions are provided that comprise methotrexate. The powder compositions described herein can be in the form of liquid crystal methotrexate, although any form (e.g., crystalline, amorphous, and so forth) of methotrexate can be used. The powder compositions can advantageously be suited for pulmonary delivery, although other uses (e.g., a powder for reconstitution) are also contemplated.

The compositions of the present invention are particularly effective for the treatment of cancers, such as lung cancer. Moreover, the spray dried methotrexate powder compositions described herein are surprising stable (i.e., exhibit minimal chemical and physical degradation upon preparation and storage, even under extreme conditions of temperature and humidity). The methotrexate powders of the invention (i) are readily dispersed by aerosol delivery devices (i.e., demonstrate good aerosol performance), (ii) exhibit surprisingly good physical and chemical stability during powder manufacture and processing, and upon storage, and (iii) are reproducibly prepared.

The methotrexate powder compositions according to the present invention comprise methotrexate, and, optionally, but not necessarily, one or more pharmaceutically acceptable excipients and/or one or more additional active agents. Advantageously, the methotrexate powder compositions can be respirable powder compositions. The components of the methotrexate powder compositions of the invention will now be described.

Methotrexate for use in the compositions described herein may be purchased from a commercial source or may be synthetically prepared according to the procedures set forth in U.S. Pat. No. 2,512,572. The chemical structure of methotrexate is provided below.

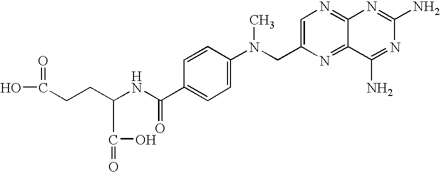

The specific methotrexate may be neutral (i.e., uncharged) or may be in the form of a pharmaceutically acceptable salt, for example, an acid addition salt such as acetate, maleate, tartrate, methanesulfonate, benzenesulfonate, toluenesulfonate, and so forth, or an inorganic acid salt such as hydrochloride, hydrobromide, sulfate, phosphate, and so on. Cationic salts may also be employed, such as salts of sodium, potassium, calcium, magnesium, or ammonium salts. Regardless of whether the methotrexate is charged, uncharged, or in a salt form, the methotrexate must be in soluble form upon administration to a patient. That is, at least some fraction of the total methotrexate amount administered must solubilize in vivo in order exert a pharmacological effect.

Similarly, any additional active agent can be present in the formulation in neutral (i.e., uncharged) form or may be in the form of a pharmaceutically acceptable organic salt, inorganic salt, and so on, as discussed in the immediately preceding paragraph.

The amount of methotrexate contained within the powder compositions will be that amount necessary to deliver a therapeutically effective amount (i.e., amount required to exert the therapeutic effect) of methotrexate per unit dose over the course of a dosing regimen. Thus, for example, for pulmonary administration, the amount is that amount necessary to deliver pharmacologically effective amount to the lungs for systemic or local delivery depending on the ultimate target site. In practice, this amount will vary depending upon the patient population, and dosing requirements. With respect to inhalation (oral or nasal), the powders are relatively disperse and losses to the inhalation device are minimized, meaning that more of the powder dose is actually delivered to the patient. This, in turn, correlates to a lower required dosage to achieve the desired therapeutic goal.

In general, the total amount of methotrexate contained in the respirable powder compositions will range from 1 to 100% of the total weight of the respirable powder composition, preferably from 5 to 98%, more preferably from 10 to 95%, even more preferably from about 45% to 95% by weight to about 50% to about 90%. A preferred dry powder composition will contain from about 40% to 80% methotrexate (% by weight of composition), and even more preferably will contain from about 0.2% to 99% methotrexate by weight.

The actual therapeutically effective amount of methotrexate required will vary from one patient to the next and from one therapeutic regimen to the next. The amount and frequency of administration will depend, of course, on factors such as the nature and severity of the indication being treated, the desired response, the patient population, condition of the patient, and so forth. Generally, a therapeutically effective amount will range from about 0.001 mg/kg/dose to 100 mg/kg/dose, preferably in doses from 0.01 mg/kg/dose to 75 mg/kg/dose, and more preferably in amounts from 0.10 mg/kg/day to 50 mg/kg/dose.

Similarly, the actual therapeutically effective amount any additional active agent can be determined by one of ordinary skill in the art and will also depend on factors such as the nature and severity of the indication being treated, the desired response, the patient population, condition of the patient, and so forth. Again, exemplary therapeutically effective amount for any additional active agent will typically range from about 0.001 mg/kg/dose to 100 mg/kg/dose, preferably in doses from 0.01 mg/kg/dose to 75 mg/kg/dose, and more preferably in amounts from 0.10 mg/kg/day to 50 mg/kg/dose.

Each dose of the final composition can be administered in a variety of dosing schedules, again depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, three times weekly, twice weekly, once weekly, twice monthly, once monthly, and any combination thereof. Once the clinical endpoint has been achieved, dosing is halted.

The respirable powder compositions of the present invention may be formulated “neat,” i.e. without pharmaceutical excipients or additives. In addition, the compositions can also be prepared to optionally include one or more additional active agents and/or one or more pharmaceutically acceptable excipients.

With respect to an optional component (e.g., one or more excipients, one or more additional active agents, and so forth) that may be present in the composition, the optional component can be present along with methotrexate in each individual particle. In such a case, the optional component can be added to the methotrexate-containing mixture prior to spray drying. Alternatively or in addition, the optional component can be prepared separately and thereafter combined (and mixed) with methotrexate-containing particles prepared as described herein.

With respect to optional additional active agents, each optional additional active agent will each independently be present in an amount ranging from about 0.01% to about 99% percent by weight, preferably from about 0.1% to about 95%, more preferably from about 0.5% to about 80%, even more preferably from about 1% to about 50-60%.

With respect to excipients, such excipients, if present, are generally present in the powder composition in amounts ranging from about 0.01% to about 99% percent by weight, preferably from about 0.1% to about 95%, more preferably from about 0.5% to about 80%, even more preferably from about 1% to about 50-60%. The Examples section describes various excipient-containing respirable methotrexate compositions. Typically, the excipient or excipients will serve to improve one or more of the following: the aerosol properties of the composition; chemical stability; physical stability; storage stability; and handling characteristics.

In particular, the excipient materials can often function to improve the physical and chemical stability of the methotrexate powder composition. For example, the excipient may minimize the residual moisture content and hinder moisture uptake and/or enhance particle size, degree of aggregation, surface properties (i.e., rugosity), ease of inhalation, and targeting of the resultant particles to the lung. The excipient(s) may also simply serve simply as bulking agents for reducing the active agent concentration in the dry powder composition.

Pharmaceutical excipients useful in the present composition include, but are not limited to, proteins (i.e., large molecules composed of one or more chains of amino acids in a specific order), oligopeptides (i.e., short chains of amino acids connected by peptide bonds), peptides (i.e., a class of molecules that hydrolyze into amino acids), amino acids, lipids (i.e., fatty, waxy or oily compounds typically insoluble in water but soluble in organic solvents, containing carbon, hydrogen and, to a lesser extent, oxygen), polymers (i.e., large molecules formed by the combination of many similar smaller molecules), and carbohydrates (e.g., sugars, including monosaccharides, di-, tri-, tetra-, and oligosaccharides; derivatized sugars such as alditols, aldonic acids, esterfied sugars and the like; and polysaccharides or sugar polymers), which may be present singly or in combination. Suitable excipients include those provided in International Publication No. WO 96/32096.

Preferred excipients include sugar alcohols, lipids, DPPC, DSPC, calcium/magnesium, amino acids (particularly hydrophobic amino acids), oligopeptides, polypeptides, and sugars (particularly hydrophobic sugars). Particularly preferred excipients include zinc salts, leucine, citrate, and sugars such as sucrose and mannitol. For particulate formulations, preferred excipients are those having glass transition temperatures (Tg), above about 35° C., preferably above about 45° C., more preferably above about 55° C.

Exemplary polypeptide and protein excipients include serum albumin such as human serum albumin (HSA), recombinant human albumin (rHA), gelatin, casein, hemoglobin, and the like. Particularly preferred are dispersibility enhancing polypeptides, e.g., HSA, as described in international Publication No. WO 96/32096.

Representative amino acid/polypeptide components, which may also function in a buffering capacity, include alanine, glycine, arginine, betaine, histidine, glutamic acid, aspartic acid, cysteine, lysine, leucine, isoleucine, valine, methionine, phenylalanine, aspartame, tyrosine, tryptophan, and the like. Preferred are amino acids and peptide that function as dispersing agents. Amino acids falling into this category include hydrophobic amino acids such as leucine (leu), valine (val), isoleucine (isoleu), tryptophan (try) alinine (ala), methionine (met), phenylalanine (phe), tyrosine (try), histidine (his), and proline (pro). One particularly preferred amino acid is the amino acid leucine. Leucine, when use in the formulations described herein, includes D-leucine, L-leucine, and racemic leucine. Dispersibility enhancing peptides for use in the invention include dimers, trimers, tetramers, and pentamers composed of hydrophobic amino acid components such as those described above. Examples include di-leucine, di-valine, di-isoleucine, di-tryptophan, di-alanine, and the like, trileucine, trivaline, triisoleucine, tritryptophan etc.; mixed di- and tri-peptides, such as leu-val, isoleu-leu, try-ala, leu-try, etc., and leu-val-leu, val-isoleu-try, ala-leu-val, and the like and homo-tetramers or pentamers such as tetra-alanine and penta-alanine. Particularly preferred oligopeptide excipients are dimers and trimers composed of 2 or more leucine residues, as described in International Patent Application PCT/US00/09785. Thus for example, preferred oligopeptides are selected from the group consisting of dileucine, leu-leu-gly, leu-leu-ala, leu-leu-val, leu-leu-leu, leu-leu-ile, leu-leu-met, leu-leu-pro, leu-leu-phe, leu-leu-trp, leu-leu-ser, leu-leu-thr, leu-leu-cys, leu-leu-tyr, leu-leu-asp, leu-leu-glu, leu-leu-lys, leu-leu-arg, leu-leu-his, leu-leu-nor, leu-gly-leu, leu-ala-leu, leu-val-leu, leu-ile-leu, leu-met-leu, leu-pro-leu, leu-phe-leu, leu-trp-leu, leu-ser-leu, leu-thr-leu, leu-cys-leu, leu-try-leu, leu-asp-leu, leu-glu-leu, leu-lys-leu, leu-arg-leu, leu-his-leu, leu-nor-leu, and combinations thereof. Of these, dileucine and trileucine are particularly preferred.

Another preferred feature of an excipient for use in the invention is surface activity. Surface active excipients, which may also function as dispersing agents, such as hydrophobic amino acids (e.g., leu, val isoleu, phe, etc.), di- and tri-peptides, polyamino acids (e.g., polyglutamic acid) and proteins (e.g., HSA, rHA, hemoglobin gelatin) are particularly preferred, since due to their surface active nature, these excipients tend to concentrate on the surface of the particles of the methotrexate composition, making the resultant particles highly dispersible in nature. Other exemplary surface active agents that may be included in the methotrexate compositions described herein include but are not limited to polysorbates, lecithin, oleic acid, benzalkonium chloride, and sorbitan esters.

Carbohydrate excipients suitable for use in the invention include, for example; monosaccharides such as fructose, maltose, galactose, glucose, d-mannose, sorbose, and the like; disaccharides, such as sucrose, raffinose, melezitose, maltodextrins, dextrans, starches and the like; and alditols, such as mannitol, xylitol, maltitol, lactitol, xylitol sorbital (glucito), myoinasitol and the like.

The respirable methotrexate compositions may also include a buffer or a pH-adjusting agent; typically, the buffer is a salt prepared from an organic acid or base. Representative buffers include organic acid salts such as salts of citric acid (to provide the corresponding citrate), ascorbic acid, gluconic acid, carbonic acid, tartaric acid, succinic acid, acetic acid, or phthalic acid, Tris, tromethamine hydrochloride, or phosphate buffer.

Additionally, the methotrexate compositions of the invention may include polymeric excipients/additives such as polyvinylpyrrolidones, derivatized celluloses such as hydroxypropylmethylcellulose, Ficcols (a polymeric sugar), hydroxyethylsartch, dextrates (e.g., cyclodextrins, such as 2-hydroxypropyl-β-cyclodextrin and sulfobutylether-β-cyclodextrin), polyethylene glycols, salts (e.g., sodium chloride), antimicrobial agents, antioxidants, antistatic agents, surfactants (e.g., polysorbates such as “TWEEN 20” and “TWEEN 80”), lecithin, oleic acid, benzalkonium chloride, sorbitan esters, lipids (e.g., phospholipids, fatty acids), steroids (e.g., cholesterol) and chelating agents (e.g., EDTA). For compositions containing a polymeric component, the polymer may typically be present to a limited extent in the composition, i.e., at levels less than about 10% by weight. Preferred compositions of the invention are those in which the methotrexate is nonliposomally or polymer encapsulated, or noncoated (i.e., absent a discrete coating layer). Preferred methotrexate compositions such as those exemplified herein are immediate-acting formulations, i.e., designed for immediate rather than for sustained release applications.

Other pharmaceutical excipients and/or additives suitable for use in the compositions according to the invention are listed in “Remington: the Science & Practice of Pharmacy,” 19^{th} ed., Williams & Williams, (1995), in the “Physician's Desk Reference,” 52^{nd} ed., Medical Economics, Montvale, N.J. (1998), and in “The Handbook of Pharmaceutical Excipients,” 3^{rd} Edition, A. H. Kibbe, ed., American Pharmaceutical Association, Pharmaceutical Press, 2000.

In accordance with the invention, the methotrexate powder composition may be a dry powder, the dry powder being crystalline, liquid crystalline, amorphous glass, or a mixture of both forms. For formulations containing a surface-active agent, the surface-active material (in either crystalline or amorphous form), will typically be present on the surface of the particles in a higher concentration than in the bulk powder.

Methotrexate powder compositions, such as respirable dry powder formulations, are preferably prepared by spray drying. Spray drying is carried out, for example, as described generally in the Spray-drying Handbook,” 5^{th} ed., K. Masters, John Wiley & Sons, Inc., NY, N.Y. (1991), and in Platz, R., et al., International Patent Publication Nos. WO 97/41833 (1997) and WO 96/32149 (1996).

Briefly, to prepare a methotrexate solution for spray drying, methotrexate [and any other excipient(s) and or active agent(s)] is generally dissolved or mixed in water, optionally containing a physiologically acceptable buffer. The pH range of solution is generally between about 3 and 10, with nearer neutral pHs being preferred, since such pHs may aid in maintaining the physiological compatibility of the powder after dissolution of powder within the lung. The aqueous formulation may optionally contain additional water-miscible solvents, such as acetone, alcohols and the like. Representative alcohols are lower alcohols such as methanol, ethanol, propanol, isopropanol, and the like. The solutions will generally contain methotrexate dissolved at a concentration from 0.01% (weight/volume) to about 20% (weight/volume), preferably from 0.1% to 10% (weight/volume), more preferably 1% to 3% (weight/volume). Alternatively, components of the methotrexate formulation may be spray dried using an organic solvent or cosolvent system, employing one or more solvents such as acetone, alcohols (e.g., methanol and ethanol), ethers, aldehydes, hydrocarbons, ketones and polar aprotic solvents.

The methotrexate-containing solutions are then spray dried in a conventional spray drier, such as those available from commercial suppliers such as Niro A/S (Denmark), Buchi (Switzerland) and the like, resulting in a methotrexate composition, preferably in the form of a respirable dry powder. Optimal conditions for spray-drying the active agent solutions will vary depending upon the formulation components, and are generally determined experimentally. The gas used to spray-dry the material is typically air, although inert gases such as nitrogen or argon are also suitable. Moreover, the temperature of both the inlet and outlet of the gas used to dry the sprayed material is such that it does not cause decomposition of the methotrexate in the sprayed material. Such temperatures are typically determined experimentally, although generally, the inlet temperature will range from about 50° C. to about 200° C. while the outlet temperature will range from about 30° C. to about 150° C.

Alternatively, although less preferably, the methotrexate powder compositions may be prepared by lyophilization, vacuum drying, spray freeze drying, super critical fluid processing, air drying, or other forms of evaporative drying. Milling and other particle-size reduction techniques can be used to provide particles in the desired range.

In some instances, it may be desirable to provide the methotrexate dry powder formulation in a form that possesses improved handling/processing characteristics, e.g., reduced static, better flowability, low caking and the like, by preparing compositions composed of fine particle aggregates, that is, aggregates or agglomerates of the above-described methotrexate. Dry powder particles, where the aggregates are readily broken back down to the fine powder components for pulmonary delivery, as described in, e.g., U.S. Pat. No. 5,654,007. Alternatively, the methotrexate powders may be prepared by agglomerating the powder components, sieving the materials to obtain the agglomerates, spheronizing to provide a more spherical agglomerate, and sizing to obtain a uniformly-sized product, as described in, e.g., International PCT Publication No. WO 95/09616.

The methotrexate dry powders are preferably maintained under dry (i.e., relatively low humidity) conditions during manufacture, processing, and storage. Irrespective of the drying process employed, the process will preferably result in respirable, highly dispersible compositions composed of substantially methotrexate particles.

Certain physical characteristics of the spray dried methotrexate powder compositions are preferred to maximize the efficiency of aerosolized delivery of such compositions to the lung.

The respirable methotrexate powder compositions are composed of particles effective to penetrate into the lungs. Passage of the particles into the lung physiology is an important aspect of the present invention. To this end, the particles of the invention have a mass median diameter (MMD) of less than about 10 μm, preferably less than 7.5 μm, and more preferably less than 5 μm, and usually are in the range of 0.1 μm to 5 μm in diameter. Preferred compositions are composed of particles having and MMD from about 0.5 to 3.5 μm. The respirable methotrexate powder compositions may also contain non-respirable carrier particles such as lactose, where the nonrespirable particles are typically greater than about 40 microns in size. In a preferred embodiment, the dry powder is non-liposomal or non-lipid containing.

The respirable methotrexate powder compositions of the invention are further characterized by an aerosol particle size distribution less than about 10 μm mass median aerodynamic diameter (MMAD), preferably less than 5.0 μm, and more preferably less than 3.5 μm. The mass median aerodynamic diameters of the powders will characteristically range from about 0.5-10 μm, preferably from about 0.5-5.0 μm MMAD, more preferably from about 1.0-4.0 μm MMAD, and even more preferably from about 1.5 to 3.5 μm.

The methotrexate powder compositions, particularly the respirable dry powder compositions, generally have a moisture content below about 10% by weight, usually below about 5% by weight, and preferably below about 3% by weight. Such low moisture-containing solids tend to exhibit a greater stability upon packaging and storage.

The dry powders preferably have a bulk density ranging from about 0.1-10 g/cc, preferably from about 0.25-4 g/cc, more preferably from about 0.5-2 g/cc, and most preferably from about 0.7-1.4 g/cc.

An additional measure for characterizing the overall aerosol performance of a dry powder is the fine particle dose or mass (FPM) or fine particle fraction (FPF), which describes the mass percentage of powder having an aerodynamic diameter less than a certain amount (e.g., 3.3 microns or 4.7 microns). Dry powders having an FPF value greater than 40% (or 0.40), more preferably greater than 50% (or 0.50), even more preferably greater than 60% (0.60) are particularly well suited for pulmonary delivery. Powders containing at least fifty percent of aerosol particles sized between 0.5 and 3.5 μm are extremely effective when delivered in aerosolized form, in reaching the regions of the lung, including the alveoli.

The spray dried methotrexate powder compositions of the present invention are “storage stable,” i.e., characterized by minimal molecular aggregate formation and/or minimal particulate aggregate formation, when stored for extended periods at extreme temperatures (“temperature stable”) and humidities (“moisture stable”). For example, the spray dried methotrexate powder compositions of the present invention experience minimal particulate aggregate formation after storage for a period of time (e.g., two weeks or more) at a temperature ranging from about 2° C. to about 50° C., preferably about 25° C., and/or a relative humidity (“RH”) ranging from 0% to about 75%, preferably about 33% RH.

It is important to note the distinctions between respirable powder-based formulations and nebulized formulations. Despite the fact that nebulized formulations may be considered by some to be “inhaleable,” in that they are breathed through the mouth and into the lungs, they are not “respirable” as defined herein. For example, nebulized formulations typically cannot reach the tissues of the deep lung. Moreover nebulized formulations are solution-based, i.e., are administered in solution rather than in solid form.

The methotrexate powder compositions, particularly the dry powder compositions described herein, are preferably delivered using any suitable dry powder inhaler (DPI), i.e., an inhaler device that utilizes the patient's inhaled breath as a vehicle to transport the previously dispersed (by passive or active means) dry powder to the lungs. Preferred dry powder inhalation devices described U.S. Pat. Nos. 5,458,135, 5,740,794, and 5,785,049, and in International Patent Publication WO 00/18084.

When administered using a device of this type, the respirable methotrexate powder composition is contained in a receptacle having a puncturable lid or other access surface, preferably a blister package or cartridge, where the receptacle may contain a single dosage unit or multiple dosage units. Large numbers of cavities are conveniently filled with metered doses of dry powder medicament as described in International Patent Publication WO 97/41031.

Also suitable for delivering the respirable methotrexate powder formulations described herein are dry powder inhalers of the type described in, for example, U.S. Pat. Nos. 3,906,950 and 4,013,075, wherein a premeasured dose of dry powder for delivery to a subject is contained within a hard gelatin capsule.

Other dry powder dispersion devices for pulmonary administration of dry powders include those described in, for example, European Patent Nos. EP 129985, EP 472598, EP 467172, and in U.S. Pat. No. 5,522,385. Also suitable for delivering the methotrexate powder compositions of the invention are inhalation devices such as the Astra-Draco “TURBUHALER.” This type of device is described in detail in U.S. Pat. Nos. 4,668,218, 4,667,668, and 4,805,811. Also suitable are devices which employ the use of a piston to provide air for either entraining powdered medicament, lifting medicament from a carrier screen by passing air through the screen, or mixing air with powder medicament in a mixing chamber with subsequent introduction of the powder to the patient through the mouthpiece of the device, such as described in U.S. Pat. No. 5,388,572.

The inhaleable methotrexate powder compositions may also be delivered using a pressurized, metered dose inhaler (MDI) containing solution or suspension of drug in a pharmaceutically inert liquid propellant, e.g., a chlorofluorocarbon or fluorocarbon, as described in U.S. Pat. Nos. 5,320,094 and 5,672,581. Prior to use, the respirable methotrexate powder compositions are generally stored in a receptacle under ambient conditions, and preferably are stored at temperatures at or below about 25° C., and relative humidities ranging from about 30 to 60%. More preferred relative humidity conditions, e.g., less than about 30%, may be achieved by the incorporation of desiccating agent in the secondary packaging of the dosage form. The respirable dry powders of the invention are characterized not only by good aerosol performance, but by good stability, as well.

When aerosolized for direct delivery to the lung, the methotrexate powder compositions described herein will exhibit good in-lung bioavailabilities.

The compositions according to the present invention are suited for a variety of uses, included, for example, the treatment of individuals suffering from cancers such as choriocarcinoma, chorioadenoma destruens, hydatidiform mole, leukemia, breast cancer, head and neck cancer, lung cancer (including squamous cell and small cell types), non-Hodgkin's lymphoma, and osteosarcoma, psoriasis, and rheumatoid arthritis.

For many types of cancers, methotrexate can be used alone or with one or more additional active agents. Examples of additional active agents include those selected from the group consisting of prednisone, prednisolone, leucovorin, dosorubicin, cisplatin, carboplatin, bleomycin, cyclophosphamide and dactinomycin.

Advantageously, the one or more additional active agents can be included in the same composition as the methotrexate, thereby resulting in a single composition or dosage form comprising two or more active agents. In such a case, an additional active agent can be present within each methotrexate particle [e.g., wherein spray-dried particles are produced from a prespray dried solution, suspension or mixture containing methotrexate and the additional active agent]. Moreover, a formulation comprising the additional active agent can be prepared separately from the methotrexate-containing particles, wherein the formulation and methotrexate-containing particles are subsequently combined.

In addition, the one or more active agents can be administered to a patient totally separate from the methotrexate formulation. Moreover, there can be a delay of time between administering the methotrexate-containing composition and the administering the additional active agent. For example, it is preferred to delay administration of leucovorin 6 hours, more preferably 12 hours, still more preferably 18 hours, and most preferably 24 hours following administration of the methotrexate-containing composition.

The following examples are illustrative of the present invention, and are not to be construed as limiting the scope of the invention. Variations and equivalents of these examples will be apparent to those of ordinary skill in the art in light of the present disclosure, the drawings and the claims herein.

All articles, books, patents, journal articles and other publications referenced herein are hereby incorporated by reference in their entirety.

### EXPERIMENTAL

Crystalline materials were defined as those having long-range three-dimensional order and amorphous solids as those lacking any long range order. Thermotropic liquid crystals present one-dimensional (nematic) or two-dimensional (smectic) order. Lyotropic liquid crystals may also display one- or two-dimensional order, as a function of the solvent content.

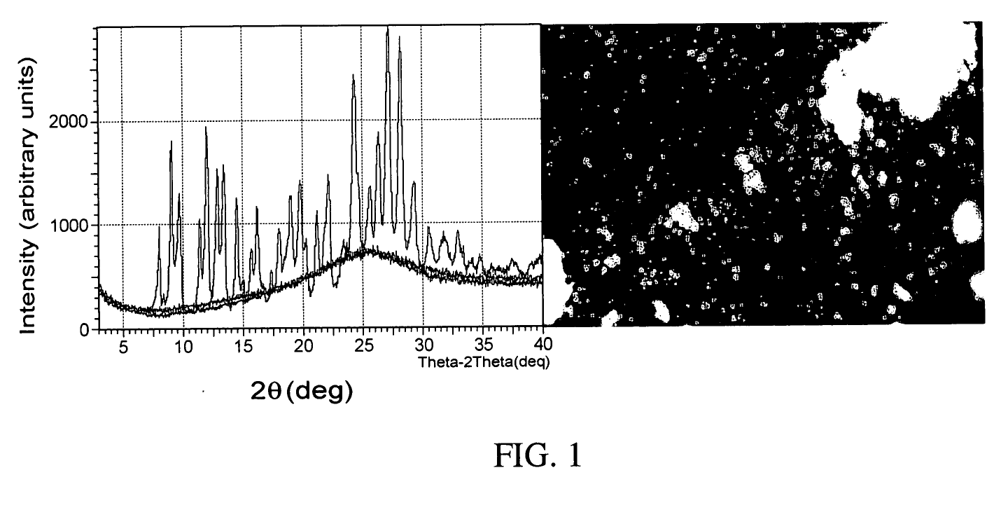

As shown below, spray dried methotrexate powder exhibited no crystalline characteristics on the XRPD patters. Birefringence under PLM was noted for the spray dried methotrexate powder (FIG. 1), indicating at least one dimensional order.

In the Examples that follow, spray dried methotrexate was either prepared or characterized. Where noted, “raw material” methotrexate or “crystalline” is compared to spray dried methotrexate.

### Example 1

### Preparation of Methotrexate Powder

Methotrexate was dissolved in aqueous alkaline solution of sodium hydroxide (1:2 molar). The methotrexate-containing solution was then spray dried using a Büchi 190 laboratory spray-dryer to form spray dried particles comprising methotrexate. Spray drying was conducted under the following conditions:

| Solution feed rate (pump): | 5 mL/minute |
|---|---|
| Inlet temperature: | 99° C. |
| Outlet temperature: | 64° C. |
| Atomizer delivery pressure: | 60 pounds/inch² |
| Venturi temperature: | 52.5° C. |
| Venturi manifold (ΔP) H₂O: | 1.8 |

The percent yield for spray drying was calculated as 23.5%.

The spray dried particles comprising methotrexate were recovered to form a methotrexate powder.

### Example 2

### Karl Fisher Moisture Determination

Moisture content in methotrexate powder was measured by Karl Fisher potentiometric titration by direct injection using a Mitsubishi model CA-06 moisturemeter (Mitsubishi Kasei Corporation, Japan). The powder was dissolved/dispersed in 2.5 mL formamide and 1 mL of the solution was injected (n+2). Titration checks were made by injecting water standards, Mitsubishi (catalogue number MC02020), at several time intervals.

The methotrexate powder prepared in Example 1 was determined to be 7.1 (0.6 % RSD, percent relative standard deviation).

### Example 3

### X-Ray Powder Diffraction (XRPD) Study

An XRPD study was performed using an XRD-6000 (Schimadzu Corporation, Japan). Samples were scanned from 3-40° 2θ, at 2°/minute, and a step size equal to 0.04°, using a Cu radiation source with a wavelength of 1.54 Å, voltage 40 kV and current 40 mA.

The methotrexate powder prepared in Example 1 exhibited no sharp peaks on XRPD patterns, suggesting no long range three dimensional order. However, one broad peak with a maximum at 26° in 2θ was observed.

### Example 4

### Polarized Light Microscopy (PLM) and Hot Stage Microscopy Study

PLM and hot stage microscopy studies were done using a Nikon (Melville, N.Y.) Optihot-2 polarizing microscope. A Mettler (Columbus, Ohio) Toledo FP82HT hot stage attachment provided the variable temperature analyses from room temperature to 250° C. at 10° C. per minute. Photomicrographs were taken using a Nikon 35 mm attachment using either a 20× or 40× objective. Microscope slides and coverslips were cleaned with methanol prior to use.

The methotrexate powder prepared in Example 1 showed strong birefringence using cross polarizers with and without the REDISPERSE brand of solvent (an organic solvent in which spray dried methotrexate) is insoluble. Birefringence persisted after quench cooling from 260° C.

### Example 5

### Small Angle X-Ray Scattering (SAXS) Study

SAXS study was performed using a Rigaku (Danvers, Mass.) 12 kW diffractometer equipped with a Kratky camera and Braun 10 cm position sensitive detector. Scan rate was 0.12° per minute from 0 to 2.2° in 2θ. Radiation was generated from a Cu source at a wavelength of 1.542 Å.

The SAXS patterns for untreated and treated methotrexate powder prepared in Example 1 (treated by quench cooling from 230° C. and 200° C., respectively) showed no characteristic peaks, indicating possibly nematic liquid crystals in all formulations. The peak observed at 0.15° is due to equipment beam stop.

### Example 6

### Dynamic Vapor Sorption (DVS) Study

Water sorption/desorption studies were conducted on a DVS-1000 moisture balance system (Surface Measurement Systems, UK). A sample (15-30 mg) was initially dried at 25° C. until a constant weight was achieved. The relative humidity was increased from 0 to 90% (with 5% relative humidity increments). Each successive increment was initiated when the change in weight at a given relative humidity was smaller than 5 μg over 5 minutes. Once the sample was exposed to 90% relative humidity, the process was reversed from 90% to 0 relative humidity (with 10% relative humidity increments).

The methotrexate powder prepared in Example 1 showed constant absorption of water to up to about 13.5% of its weight at 90% relative humidity and no hysteresis upon desorption of water, indicating no moisture-induced state change. Spray dried methotrexate sodium was shown hygroscopic than the crystalline material. In addition, signs of moisture-induced crystallization are observed in the DVS time profile at 70% relative humidity. The crystalline species formed is possibly a crystalline hydrate of methotrexate sodium consistent with the hysteresis. DVS data suggest that the crystalline hydrate has 13% of its weight water. DVS data also suggest that long term stability at 25° C. may be achieved if the liquid crystalline phase is stored below 70% relative humidity.

### Example 7

### Scanning Electron Microscopy (SEM) Study

SEM was utilized to observe morphology of the spray dried particles confirmed to be in the liquid crystalline state as well as the starting materials. Samples were mounted on silicon wafers mounted on top of double-sided carbon tape on an aluminum SEM stub. The same was then sputter coated in a Denton sputter coater for 60-90 seconds at 75 mTorr and 42 mA with gold:palladium. Images were taken with a Philips XL30 ESEM operated in high vacuum mode using an Everhart-thornley detector to capture secondary electrons for the image composition. Accelerating voltage was set at 20 kV using a LaB₆ source. Working distance was 5-6 mm.

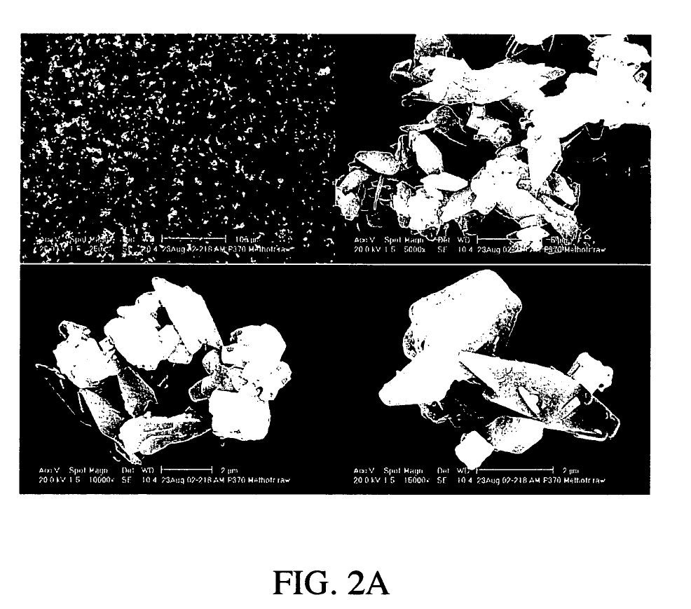

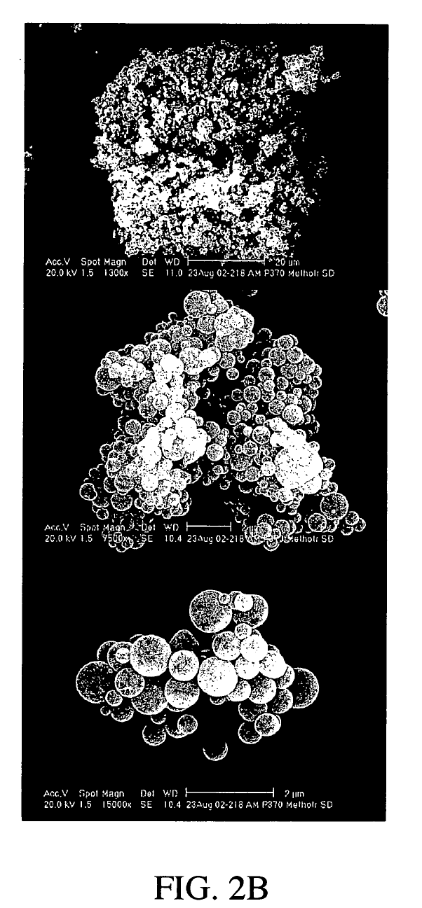

Crystalline methotrexate showed orthorhombic-shaped crystals with relatively sharp edges, as well as a few cubic-shaped particles (FIG. 2A). The methotrexate powder prepared in Example 1 showed perfectly spherical particles (FIG. 2B) with a relatively narrow particle size distribution.

### Example 8

### Modulated Differential Scanning Calorimetry (MDSC) Study

MDSC studies were performed using a TA 2920 instrument (TA instruments, New Castle, Del.). A sample (5-10 mg) of the powder was filled into an aluminum DSC pan in a glove box, with the relative humidity maintained under 1%, and hermetically sealed using a sample encapsulation press. Helium was used as a purge at 30 cm³/minute; the Refrigerated Control System (RCS) used helium at a flow rate of 110 cm³/minute. The MDSC protocol was as follows: equilibrate at 0° C.; modulate ±0.318° C. every 60 seconds; ramp 2° C./minute to 200° C.; isothermal for 5 minutes; ramp 10° C./minute to 0° C.; isothermal for 5 minutes; modul 0.318° C. every 60 seconds; ramp 2° C./minute to 280° C.

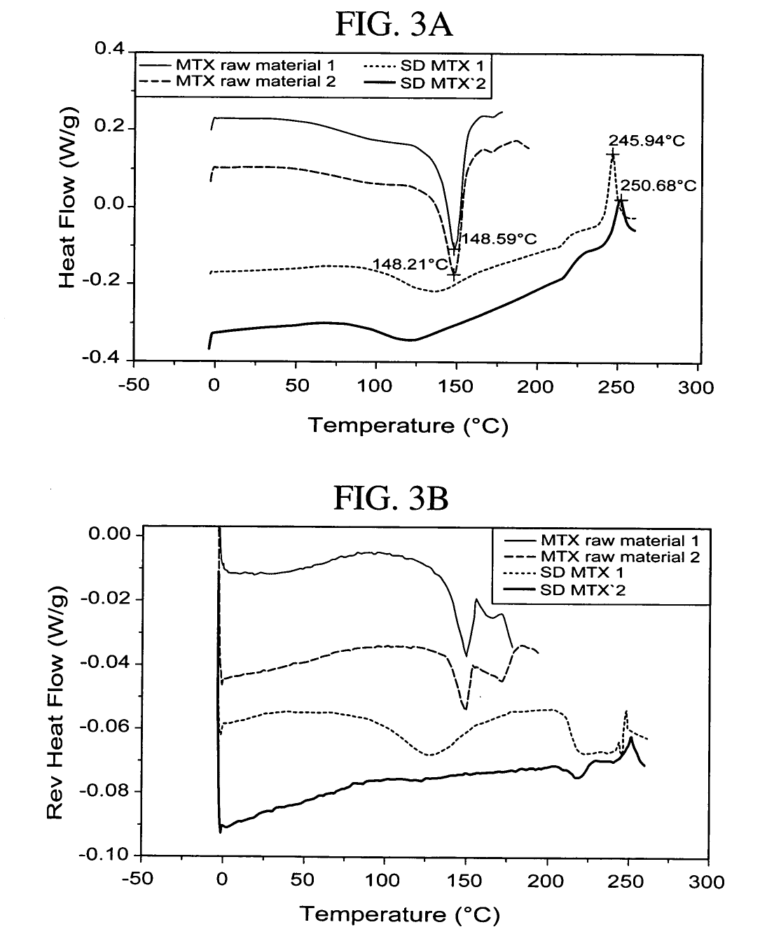

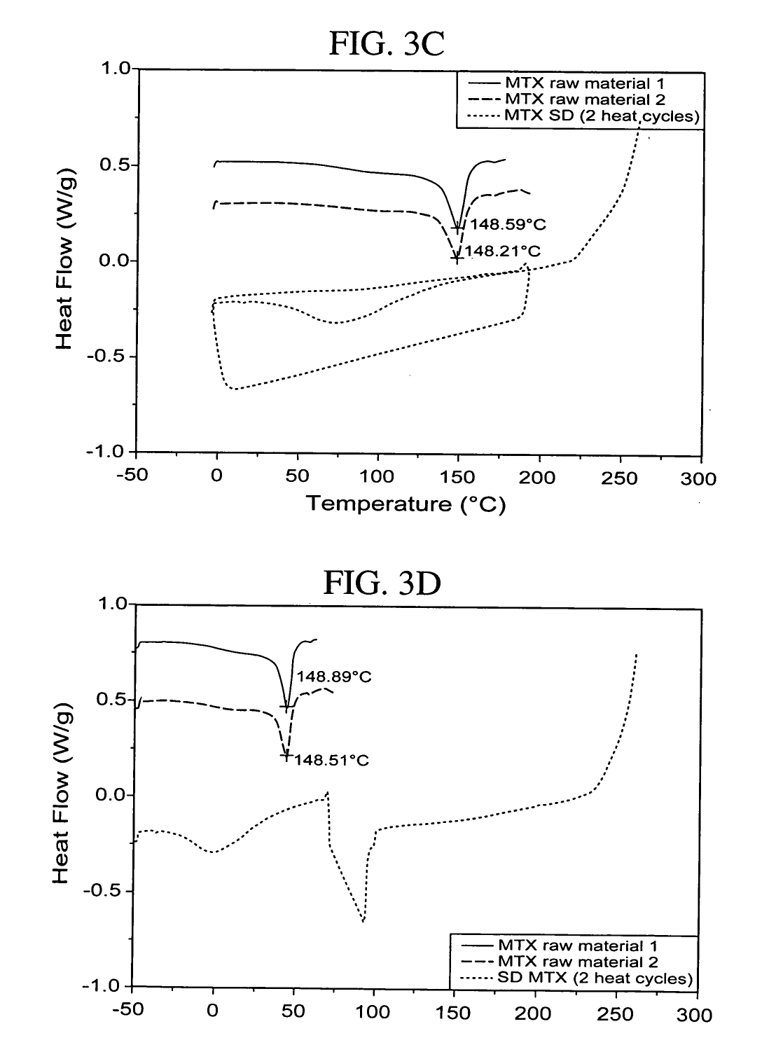

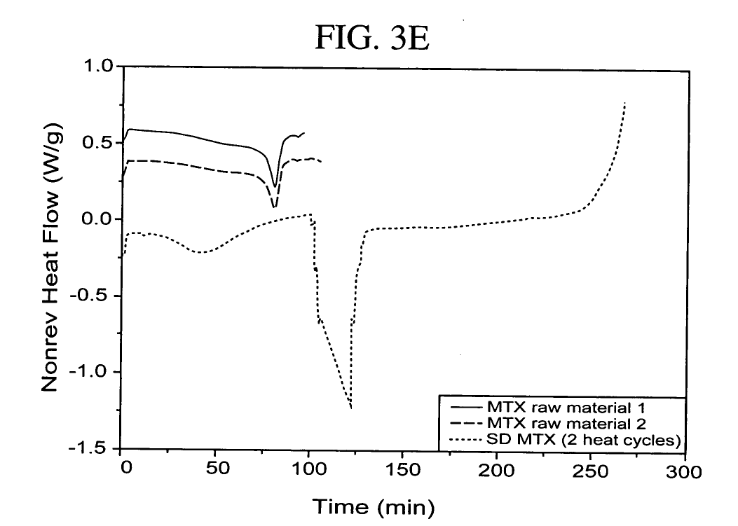

Crystalline methotrexate showed a melting point of 148.7±0.3° C. MDSC thermogram for the methotrexate powder prepared in Example 1 showed a very broad endotherm starting at 25° C. This endotherm disappeared in the second heating cycle. The spray dried material decomposed at 246-250° C. No Tg (glass transition temperature) was observed in both heating cycles. See FIGS. 3A, 3B and 3C for MDSC data.

1. A pharmaceutical formulation comprising plurality of spray dried particles, the particles comprising methotrexate in a liquid crystal state.

2. The pharmaceutical formulation of claim 1 suitable for delivery by inhalation.

3. The pharmaceutical formulation of claim 2 wherein the particles have a mass median diameter of less than 10 μM.

4. The pharmaceutical formulation of claim 3 wherein the particles have a mass median diameter of less than 5 μM.

5. The pharmaceutical formulation of claim 2 wherein the particles have a median aerodynamic diameter (MMAD) of less than 10 μM.

6. The pharmaceutical formulation of claim 5 wherein the particles have a median aerodynamic diameter (MMAD) of less than 5 μM.

7. The pharmaceutical formulation of claim 2 additionally comprising a pharmaceutically acceptable excipient.

8. The pharmaceutical formulation of claim 7 wherein the excipients include at least one compound selected from the group consisting of an amino acid, a peptide, a carbohydrate, a phospholipids, a lipid, and the salt of an organic acid.

9. The pharmaceutical formulation of claim 7 wherein the excipients include at least one compound selected from the group consisting of sucrose, mannitol, galactose, mannose, sorbose, lactose, trehalose, cyclodextrin, raffinose, maltodextrin, dextran, xylitol, a citrate salt, leucine, trileucine, human serum albumin and combinations thereof.

10. The pharmaceutical formulation of claim 7 wherein the pharmaceutically acceptable excipient and the methotrexate are substantially both co-localized within the same particles.

11. The pharmaceutical formulation of claim 7 wherein the pharmaceutically acceptable excipient and the methotrexate are substantially not co-localized within the same particles, but are present in different particles.

12. The pharmaceutical formulation of claim 11 wherein the excipient particles and the methotrexate particles are of different mean diameters.

13. The composition of claim 2, containing less than 5% degradation products.

14. A method for preparing a formulation, the formulation comprising a liquid crystal form of methotrexate, the method comprising the steps of: (i) combining methotrexate, an optional excipient, and solvent to form a mixture; and (ii) spray drying the mixture or solution to obtain dry particles comprised of methotrexate in a liquid crystal form and the excipient, when present.

15. The method of claim 14, wherein the spray dried formulation contains less than 5% degradation products.

16. The method of claim 14, wherein the mixture is an aqueous solution.

17. The method of claim 14, wherein the spray dried powder comprises particles 98% or more of which have a diameter of 10 microns or less.

18. The method of claim 14, wherein the spray dried powder comprises particles about 90% of which have a diameter of 5 microns or less.

19. A method for administering a formulation comprising a liquid crystal form of methotrexate to the lungs of a patient, the method comprising the steps of: (i) providing a formulation of claim 4; (ii) dispersing the formulation to form an aerosol; and (iii) delivering the aerosol to the lungs of the patient by inhalation of the aerosol by the patient.

20. The method of claim 19 wherein delivering the aerosol to the lungs is performed employing a metered dose inhaler.
